(19) United States
(12) Patent Application Publication
(10) Pub. No.: US 2012/0207717 A1
(43) Pub. Date: Aug. 16, 2012

(54) Methods for treating inhalation injury

(76) Inventor: George L. Sing, New York, NY (US)

(21) Appl. No.: 13/385,239

(22) Filed: Feb. 9, 2012

Related U.S. Application Data
(60) Provisional application No. 61/463,183, filed on Feb. 14, 2011.

(51) Int. Cl. A61K 38/19; A61P 11/00; A61K 35/48

(52) U.S. Cl. 424/93.7; 514/8.1

(57) ABSTRACT

The invention is directed to methods for treating inhalation injuries. Such methods utilize novel cell compositions such extraembryonic cytokine secreting (ECS) cells and Amnion-derived Multipotent Progenitor (AMP) cells and novel cellular factor-containing solution compositions such as extraembryonic cell-derived cellular cytokine solution, Amnion-derived Cellular Cytokine Solution (ACCS), and physiologic cytokine solution (PCS) compositions. The compositions may be used alone or in combination with each other and/or other agents.

### CROSS-REFERENCE TO RELATED APPLICATIONS

This application claims priority under 35 USC §119(e) of U.S. Provisional Application No. 61/463,183, filed Feb. 14, 2011, the entirety of which is incorporated herein by reference.

### FIELD OF THE INVENTION

The field of the invention is directed to methods of treating inhalation injuries. Such methods utilize novel cell compositions such extraembryonic cytokine secreting (ECS) cells, Amnion-derived Multipotent Progenitor (AMP) cells, including novel aerosolized ECS and AMP cells, novel cellular factor-containing solution (CFS) compositions such as extraembryonic cell-derived cellular cytokine solution, Amnion-derived Cellular Cytokine Solution (ACCS), and physiologic cytokine solution (PCS) compositions, including novel aerosolized cellular factor-containing solution compositions such as extraembryonic cell-derived cellular cytokine solution, ACCS and PCS compositions. The compositions may be used alone or in combination with each other and/or other agents.

### BACKGROUND OF THE INVENTION

The human respiratory system is situated in the thorax, and is responsible for exchange of respiratory gases (oxygen (O₂) and carbon dioxide (CO₂)) between the circulatory system and the external environment. Air is inhaled via the upper conducting airways (the nasal cavity, pharynx and larynx) through the lower conducting airways (trachea, primary bronchi and bronchial tree) and into the small bronchioles and alveoli where respiratory exchange occurs. The lungs are divided into lobes with the left lung having two lobes and the right lung having three lobes.

Histologically, the entire respiratory system is lined with simple epithelia which are supported by an underlying parenchyma. The trachea and bronchi are lined with pseudostratified ciliated columnar epithelium (PSCCE) which contains goblet cells and sero-mucous glands. The bronchioles are lined with ciliated simple columnar or cuboidal epithelium and sero-mucous glands. The respiratory bronchioles are lined with ciliated simple cuboidal epithelium. Finally, the alveoli are made of flattened Type I pneumocytes which are specialized for gas exchange, and Type II pneumocytes which secrete surfactant.

Fire has been associated with three different types of inhalation injuries to the respiratory system. They are damage from heat inhalation, damage from the inhalation of systemic toxins and damage from smoke inhalation. When inhalation injuries are combined with external burns the chance of death can increase significantly. In fact, most death associated with fire is due to inhalation injury.

Evidence that inhalation injury has occurred usually appears within 2-48 hours after the burn. Symptoms may include fainting, respiratory distress or upper airway obstruction, soot around the mouth or nose, singed nasal hairs, eyebrows and eyelashes, and burns around the face or neck. Upper airway edema is often the earliest consequence of inhalation injury and is commonly seen during the first 6 to 24 hours after injury. Generally, obstruction of the upper airway is managed with intubation and initial treatment consists of removing the patient from the gas and allowing him to breathe air or O₂.

True lung (thermal) burn occurs only if a person directly breathes in a hot air or a flame source or if high pressure forces the heat into the person. In most cases, thermal injury is confined to the upper airways, because the trachea usually protects the lung from the heat. However, lower airway injury can occur after inhalation of steam as it has a greater thermal capacity than dry air and its temperature is not adequately modulated by the upper airways the way hot dry air is.

Smoke inhalation is frequently hidden or overlooked because of more obvious visible injuries such as burns. This often leads to the victim not receiving the appropriate medical attention because the rescuers are attending to the more apparently injured victims. In fact, victims that appear apparently unharmed often collapse due to severe smoke inhalation, and 60% to 80% of fatalities resulting from burn injuries can be attributed to smoke inhalation. Smoke generally contains one or more irritant gases as well as systemic toxins such as CO. For example, the irritant gas sulfur dioxide is generated by the combustion of coal, oil and cooking fuel. It can also be generated by smelting. Sulfur dioxide causes upper airway epithelial damage. The irritant gas nitrogen dioxide is generated by the combustion of diesel and is also generated during welding and in the manufacturing of dyes, lacquers and wall paper. Nitrogen dioxide causes terminal airway epithelial damage. Other irritant gases that can cause inhalation injury include ammonia, which can be generated from fertilizer, refrigerant, in the manufacture of dyes, plastics and nylon, and can cause upper airway epithelial damage; and chlorine from bleaching agents and disinfecting products which can cause lower airway epithelia damage.

Systemic toxins are inhaled toxins that affect the body's ability to absorb O₂. If someone is found unconscious or acting confused near an enclosed fire, inhalation of systemic toxins could be the cause. However, in other instances, the person may appear symptomless. For example, carbon monoxide (CO) poisoning appears symptomless up until the point where the victim falls into a coma. In many cases, systemic toxin poisoning will cause permanent damage to organs including the brain.

Current therapies for treating inhalation injuries, including burns, include intubation, administration of steroids, and administration of antibiotics. The subject invention described herein provides novel methods for treating and accelerating the healing of airway epithelia and parenchyma damaged by inhalation burns. Such novel methods utilize the novel compositions described herein.

### SUMMARY OF THE INVENTION

It is an object of the instant invention to provide methods for treating inhalation injury, particularly inhalation burns and injuries caused by irritant gases, by administering novel cell compositions and/or novel cellular factor-containing solution (CFS) compositions. Such treatment accelerates the healing of damaged airway epithelia and parenchyma. Because the cellular factors secreted by the cells of the invention and contained in the CFS compositions are present in levels comparable to physiological levels found in the body, they are optimal for use in therapeutic applications which require intervention to support, initiate, replace, accelerate or otherwise influence biochemical and biological processes involved in the treatment and/or accelerated healing of injured tissue such as respiratory tissue injured by inhalation of heat and/or irritant gases.

Accordingly, a first aspect of the invention is a method for treating inhalation injuries in a subject in need thereof comprising administering to the subject an effective amount of a composition selected from the group consisting of extraembryonic cytokine-secreting (ECS) cells, extraembryonic cell-derived cellular cytokine solution, physiologic cytokine solution (PCS), or a combination thereof.

In one embodiment of the invention, the inhalation injury is a thermal burn. In a specific embodiment the thermal burn is caused by heat or steam. In another embodiment, the inhalation injury is caused by an irritant gas. In a specific embodiment the irritant gas of is selected from the group consisting of sulfur dioxide, nitrogen dioxide, ammonia and chlorine. In yet another embodiment the inhalation injury is smoke inhalation.

In another embodiment the extraembryonic cell-derived cellular cytokine solution is Amnion-derived Cellular Cytokine Solution (ACCS). In a specific embodiment, the ACCS comprises physiologic concentrations of VEGF, TGFβ2, Angiogenin, PDGF, TIMP-1 and TIMP-2.

Another embodiment is a therapeutic component consisting essentially of physiologic concentrations of VEGF, TGFβ2, Angiogenin, PDGF, TIMP-1 and TIMP-2, and a carrier, wherein the carrier is normal saline, PBS, lactated Ringer's solution or cell culture medium.

Still another particular embodiment is one in which the ECS cells are Amnion-derived Multipotent Progenitor (AMP) cells.

In another embodiment the compositions are aerosolized. And in a specific embodiment the aerosolized compositions are administered by nebulizer, vaporizer, or atomizer.

### DEFINITIONS

As defined herein “isolated” refers to material removed from its original environment and is thus altered “by the hand of man” from its natural state.

As used herein, the term “protein marker” means any protein molecule characteristic of the plasma membrane of a cell or in some cases of a specific cell type.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

The term “placenta” as used herein means both preterm and term placenta.

As used herein, the term “totipotent cells” shall have the following meaning. In mammals, totipotent cells have the potential to become any cell type in the adult body; any cell type(s) of the extraembryonic membranes (e.g., placenta). Totipotent cells are the fertilized egg and approximately the first 4 cells produced by its cleavage.

As used herein, the term “pluripotent stem cells” shall have the following meaning. Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploid.

As used herein, the term “multipotent stem cells” are true stem cells but can only differentiate into a limited number of types. For example, the bone marrow contains multipotent stem cells that give rise to all the cells of the blood but may not be able to differentiate into other cells types.

As used herein, the term “extraembryonic tissue” means tissue located outside the embryonic body which is involved with the embryo's protection, nutrition, waste removal, etc. Extraembryonic tissue is discarded at birth. Extraembryonic tissue includes but is not limited to the amnion, chorion (trophoblast and extraembryonic mesoderm including umbilical cord and vessels), yolk sac, allantois and amniotic fluid (including all components contained therein). Extraembryonic tissue and cells derived therefrom have the same genotype as the developing embryo.

As used herein, the term “extraembryonic cytokine-secreting cells” or “ECS cells” means a population of cells derived from the extraembryonic tissue which have the characteristic of secreting VEGF, Angiogenin, PDGF and TGFβ2 and the MMP inhibitors TIMP-1 and/or TIMP-2 at physiologically relevant levels in a physiologically relevant temporal manner into the extracellular space or into the surrounding culture media. ECS cells have not been cultured in the presence of any non-human animal materials, making them and cell products derived from them suitable for human clinical use as they are not xeno-contaminated. ECS cells may be selected from populations of cells and compositions described in this application and in US2003/0235563, US2004/0161419, US2005/0124003, U.S. Provisional Application Nos. 60/666,949, 60/699,257, 60/742,067, 60/813,759, U.S. application Ser. No. 11/333,849, U.S. application Ser. No. 11/392,892, PCTUS06/011392, US2006/0078993, PCT/US00/40052, U.S. Pat. No. 7,045,148, US2004/0048372, and US2003/0032179, the contents of which are incorporated herein by reference in their entirety.

As used herein, the term “Amnion-derived Multipotent Progenitor cell” or “AMP cell” means a specific population of cells that are epithelial cells derived from the amnion. AMP cells have the following characteristics. They have not been cultured in the presence of any non-human animal materials, making them and cell products derived from them suitable for human clinical use as they are not xeno-contaminated. AMP cells are cultured in basal medium supplemented with human serum albumin. In a preferred embodiment, the AMP cells secrete the cytokines VEGF, Angiogenin, PDGF and TGFβ2 and the MMP inhibitors TIMP-1 and/or TIMP-2. The physiological range of the cytokine or cytokines in the unique combination is as follows: ˜5-16 ng/mL for VEGF, ˜3.5-4.5 ng/mL for Angiogenin, ˜100-165 pg/mL for PDGF, ˜2.5-2.7 ng/mL for TGFβ2, ˜0.68 μg/mL for TIMP-1 and ˜1.04 μg/mL for TIMP-2. The AMP cells may optionally express Thymosin β4. AMP cells grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion-derived cells, from which AMP cells are isolated, will not react with antibodies to the stem/progenitor cell markers c-kit (CD117) and Thy-1 (CD90). Several procedures used to obtain cells from full term or pre-term placenta are known in the art (see, for example, US 2004/0110287; Anker et al., 2005, Stem Cells 22:1338-1345; Ramkumar et al., 1995, Am. J. Ob. Gyn. 172:493-500). However, the methods used herein provide improved compositions and populations of cells.

By the term “animal-free” when referring to certain compositions, growth conditions, culture media, etc. described herein, is meant that no non-human animal-derived materials, such as bovine serum, proteins, lipids, carbohydrates, nucleic acids, vitamins, etc., are used in the preparation, growth, culturing, expansion, storage or formulation of the certain composition or process. By “no non-human animal-derived materials” is meant that the materials have never been in or in contact with a non-human animal body or substance so they are not xeno-contaminated. Only clinical grade materials, such as recombinantly produced human proteins, are used in the preparation, growth, culturing, expansion, storage and/or formulation of such compositions and/or processes.

By the term “serum-free” when referring to certain compositions, growth conditions, culture media, etc. described herein, is meant that no non-human animal-derived serum is used in the derivation, preparation, growth, culturing, expansion, storage or formulation of the certain composition or process.

By the term “expanded”, in reference to cell compositions, means that the cell population constitutes a significantly higher concentration of cells than is obtained using previous methods. For example, the level of cells per gram of amniotic tissue in expanded compositions of AMP cells is at least 50 and up to 150 fold higher than the number of cells in the primary culture after 5 passages, as compared to about a 20 fold increase in such cells using previous methods. In another example, the level of cells per gram of amniotic tissue in expanded compositions of AMP cells is at least 30 and up to 100 fold higher than the number of cells in the primary culture after 3 passages. Accordingly, an “expanded” population has at least a 2 fold, and up to a 10 fold, improvement in cell numbers per gram of amniotic tissue over previous methods. The term “expanded” is meant to cover only those situations in which a person has intervened to elevate the number of the cells.

As used herein, the term “passage” means a cell culture technique in which cells growing in culture that have attained confluence or are close to confluence in a tissue culture vessel are removed from the vessel, diluted with fresh culture media (i.e. diluted 1:5) and placed into a new tissue culture vessel to allow for their continued growth and viability. For example, cells isolated from the amnion are referred to as primary cells. Such cells are expanded in culture by being grown in the growth medium described herein. When such primary cells are subcultured, each round of subculturing is referred to as a passage. As used herein, “primary culture” means the freshly isolated cell population.

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium. Examples of methods of preparing conditioned media are described in U.S. Pat. No. 6,372,494 which is incorporated by reference in its entirety herein. As used herein, conditioned medium also refers to components, such as proteins, that are recovered and/or purified from conditioned medium or from ECS cells, including AMP cells.

As used herein, the term “cellular factor-containing solution” or “CFS” composition means a composition having physiologic concentrations of one or more factors selected from VEGF, Angiogenin, PDGF and TGFβ2 and at least one MMP inhibitor. Examples of suitable MMP inhibitors include but are not limited to TIMP-1 and TIMP-2. CFS compositions include conditioned media derived from ECS cells, ACCS compositions (see definition below), physiologic cytokine solution compositions (see definition below), and sustained release formulations and aerosolized formulations of such CFS compositions.

As used herein, the term “Amnion-derived Cellular Cytokine Solution” or “ACCS” means conditioned medium that has been derived from AMP cells that have been cultured in basal media supplemented with human serum albumin.

As used herein, the term “physiologic cytokine solution” or “PCS” composition means a composition which is not cell-derived and which has physiologic concentrations of one or more factors selected from VEGF, Angiogenin, PDGF and TGFβ2 and at least one MMP inhibitor. Examples of suitable MMP inhibitors include but are not limited to TIMP-1 and TIMP-2. Details on PCS can be found in U.S. Publication No. US-2009-0054339-A1, the contents of which are incorporated herein by reference.

As used herein, the term “solution” as used in “Amnion-derived Cellular Cytokine Solution” means a liquid containing dispersed components, i.e. cytokines. The dispersed components may be fully solubilized, partially solubilized, suspended or otherwise dispersed in the liquid. Suitable liquids include, but are not limited to, water, osmotic solutions such as salt and/or sugar solutions, cell culture media, and other aqueous or non-aqueous solutions.

As used herein, the term “aerosol” means a cloud of solid or liquid particles in a gas.

The terms “particles”, “aerosolized particles”, and “aerosolized particles of formulation” are used interchangeably herein and shall mean particles of formulation comprised of any pharmaceutically active ingredient, preferably in combination with a carrier, (e.g., a pharmaceutically active respiratory drug and carrier). The particles have a size which is sufficiently small such that when the particles are formed they remain suspended in the air or gas for a sufficient amount of time such that a patient can inhale the particles into the patient's lungs.

As used herein, the term “nebulizer” means a device used to reduce a liquid medication to extremely fine cloudlike particles (i.e. an aerosol). A nebulizer is useful in delivering medication to deeper parts of the respiratory tract. Nebulizers may also be referred to as atomizers and vaporizers.

The term “lysate” as used herein refers to the composition obtained when cells, for example, AMP cells, are lysed and optionally the cellular debris (e.g., cellular membranes) is removed. This may be achieved by mechanical means, by freezing and thawing, by sonication, by use of detergents, such as EDTA, or by enzymatic digestion using, for example, hyaluronidase, dispase, proteases, and nucleases. In some instances, it may be desirable to lyse the cells and retain the cellular membrane portion and discard the remaining portion of the lysed cells.

The term “physiologic” or “physiological level” as used herein means the level that a substance in a living system is found and that is relevant to the proper functioning of a biochemical and/or biological process.

As used herein, the term “pooled” means a plurality of compositions that have been combined to create a new composition having more constant or consistent characteristics as compared to the non-pooled compositions. For example, pooled ACCS has more constant or consistent characteristics compared to non-pooled ACCS. Examples of pooled compositions include “SP pools” (more than one ACCS collection/one placenta), “MP1 pools” (one ACCS collection/placenta, multiple placentas), and “MP2 pools” (more than one ACCS collection/placenta, multiple placentas).

As used herein, the term “substrate” means a defined coating on a surface that cells attach to, grown on, and/or migrate on. As used herein, the term “matrix” means a substance that cells grow in or on that may or may not be defined in its components. The matrix includes both biological and non-biological substances. As used herein, the term “scaffold” means a three-dimensional (3D) structure (substrate and/or matrix) that cells grow in or on. It may be composed of biological components, synthetic components or a combination of both. Further, it may be naturally constructed by cells or artificially constructed. In addition, the scaffold may contain components that have biological activity under appropriate conditions.

The term “cell product” or “cell products” as used herein refers to any and all substances made by and secreted from a cell, including but not limited to, protein factors (i.e. growth factors, differentiation factors, engraftment factors, cytokines, morphogens, proteases (i.e. to promote endogenous cell delamination, protease inhibitors), extracellular matrix components (i.e. fibronectin, etc.).

The term “therapeutically effective amount” means that amount of a therapeutic agent necessary to achieve a desired physiological effect (i.e. accelerate inhalation injury healing).

As used herein, the term “pharmaceutically acceptable” means that the components, in addition to the therapeutic agent, comprising the formulation, are suitable for administration to the patient being treated in accordance with the present invention.

As used herein, the term “therapeutic component” means a component of the composition which exerts a therapeutic benefit when the composition is administered to a subject.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

As used herein, the term “tissue” refers to an aggregation of similarly specialized cells united in the performance of a particular function.

As used herein, the terms “a” or “an” means one or more; at least one.

As used herein, the term “adjunctive” means jointly, together with, in addition to, in conjunction with, and the like.

As used herein, the term “co-administer” can include simultaneous or sequential administration of two or more agents.

As used herein, the term “agent” means an active agent or an inactive agent. By the term “active agent” is meant an agent that is capable of having a physiological effect when administered to a subject. Non-limiting examples of active agents include growth factors, cytokines, antibiotics, cells, conditioned media from cells, anti-inflammatory agents, steroids, etc. By the term “inactive agent” is meant an agent that does not have a physiological effect when administered. Such agents may alternatively be called “pharmaceutically acceptable excipients”. Non-limiting examples include time release capsules and the like.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, epidural, intracerebral and infrasternal injection or infusion.

The terms “sustained-release”, “extended-release”, “time-release”, “controlled-release”, or “continuous-release” as used herein means an agent, typically a therapeutic agent or drug, that is formulated to dissolve slowly and be released over time.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

As used herein, a “wound” is any disruption, from whatever cause, of normal anatomy (internal and/or external anatomy) including but not limited to traumatic injuries such as mechanical (i.e. contusion, penetrating), thermal, chemical, electrical, radiation, concussive and incisional injuries; elective injuries such as operative surgery and resultant incisional hernias, fistulas, etc.; acute wounds, chronic wounds, infected wounds, and sterile wounds, as well as wounds associated with disease states (i.e. ulcers caused by diabetic neuropathy or ulcers of the gastrointestinal or genitourinary tract). A wound is dynamic and the process of healing is a continuum requiring a series of integrated and interrelated cellular processes that begin at the time of wounding and proceed beyond initial wound closure through arrival at a stable scar. These cellular processes are mediated or modulated by humoral substances including but not limited to cytokines, lymphokines, growth factors, and hormones. In accordance with the subject invention, “wound healing” refers to improving, by some form of intervention, the natural cellular processes and humoral substances of tissue repair such that healing is faster, and/or the resulting healed area has less scaring and/or the wounded area possesses tissue strength that is closer to that of uninjured tissue and/or the wounded tissue attains some degree of functional recovery.

As used herein, the term “inhalation injury” means an injury to the respiratory system caused by heat inhalation, damage from the inhalation of irritant gases, damage from the inhalation of systemic toxins, or damage from smoke inhalation.

As used herein the term “standard animal model for inhalation injury” refers to any art-accepted animal model for inhalation injury healing in which the compositions of the invention exhibit efficacy as measured by accelerated healing of inhalation injury.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Obtaining and Culturing of Cells

Obtaining and Culturing of Cells

ECS cells—Various methods for isolating cells from extraembryonic tissue, which may then be used to produce the ECS cells of the instant invention are described in the art (see, for example, US2003/0235563, US2004/0161419, US2005/0124003, U.S. Provisional Application Nos. 60/666,949, 60/699,257, 60/742,067, 60/813,759, U.S. application Ser. No. 11/333,849, U.S. application Ser. No. 11/392,892, PCTUS06/011392, US2006/0078993, PCT/US00/40052, U.S. Pat. No. 7,045,148, US2004/0048372, and US2003/0032179).

Identifying ECS cells—Once extraembryonic tissue is isolated, it is necessary to identify which cells in the tissue have the characteristics associated with ECS cells (see definition above). For example, cells are assayed for their ability to secrete VEGF, Angiogenin, PDGF and TGFβ2 and the MMP inhibitors TIMP-1 and/or TIMP-2 into the extracellular space or into surrounding culture media. In some instances, it may be difficult or impossible to detect certain factors using standard assays. This may be because certain factors are secreted by the cells at physiological levels that are below the level of detection by the assay methods. It may also be that the factor(s) is being utilized by the ECS cell and/or by other local cells, thus preventing accumulation at detectable levels using standard assays. It is also possible that the temporal manner in which the factors are secreted may not coincide with the timing of sampling.

AMP cell compositions are prepared using the steps of a) recovery of the amnion from the placenta, b) dissociation of the epithelial cells from the amniotic membrane using a protease, c) culturing of the cells in a basal medium with the addition of a naturally derived or recombinantly produced human protein (i.e. human serum albumin) and no non-human animal protein; d) selecting AMP cells from the epithelial cell culture, and optionally e) further proliferation of the cells, optionally using additional additives and/or growth factors (i.e. recombinant human EGF). Details are contained in US Publication No. 2006-0222634-A1, which is incorporated herein by reference.

Culturing of the AMP cells—The cells are cultured in a basal medium. Such medium includes, but is not limited to, EPILIFE® culture medium for epithelial cells (Cascade Biologicals), OPTI-PROT™ serum-free culture medium, VP-SFM serum-free medium, IMDM highly enriched basal medium, KNOCKOUTT™ DMEM low osmolality medium, 293 SFM II defined serum-free medium (all made by Gibco; Invitrogen), HPGM hematopoietic progenitor growth medium, Pro 293S-CDM serum-free medium, Pro 293A-CDM serum-free medium, UltraMDCK™ serum-free medium (all made by Cambrex), STEMLINE® T-cell expansion medium and STEMLINE® II hematopoietic stem cell expansion medium (both made by Sigma-Aldrich), DMEM culture medium, DMEM/F-12 nutrient mixture growth medium (both made by Gibco), Ham's F-12 nutrient mixture growth medium, M199 basal culture medium (both made by Sigma-Aldrich), and other comparable basal media. Such media should either contain human protein or be supplemented with human protein. As used herein a “human protein” is one that is produced naturally or one that is produced using recombinant technology. “Human protein” also is meant to include a human derivative or preparation thereof, such as human serum, which contains human protein. In specific embodiments, the basal media is IMDM highly enriched basal medium, STEMLINE® T-cell expansion medium or STEMLINE® II hematopoietic stem cell expansion medium, or OPTI-PRO™ serum-free culture medium, or combinations thereof and the human protein is human serum albumin is at least 0.5% and up to 10%. In particular embodiments, the human serum albumin is from about 0.5 to about 2%. In a specific embodiment the human albumin is at 0.5%. The human albumin may come from a liquid or a dried (powder) form and includes, but is not limited to, recombinant human serum albumin, PLASBUMIN® normal human serum albumin and PLASMANATE® human blood fraction (both made by Talecris Biotherapeutics).

In a most preferred embodiment, the cells are cultured using a system that is free of non-human animal products to avoid xeno-contamination. In this embodiment, the culture medium is IMDM highly enriched basal medium, STEMLINE® T-cell expansion medium or STEMLINE® II hematopoietic stem cell expansion medium, OPTI-PRO™ serum-free culture medium, or DMEM culture medium, with human serum albumin (i.e. PLASBUMIN® normal human serum albumin) added up to amounts of 10%.

The invention further contemplates the use of any of the above basal media wherein animal-derived proteins are replaced with recombinant human proteins and animal-derived serum, such as BSA, is replaced with human serum albumin. In preferred embodiments, the media is serum-free in addition to being animal-free.

Optionally, other factors are used. In one embodiment, epidermal growth factor (EGF) at a concentration of between 0-1 μg/mL is used. In a preferred embodiment, the EGF concentration is around 10-20 ng/mL. All supplements are clinical grade.

Generation of CFC, Including ACCS

ECS conditioned medium—is obtained as described below for ACCS, except that ECS cells are used.

Generation of ACCS—The AMP cells of the invention can be used to generate ACCS. In one embodiment, the AMP cells are isolated as described herein and 1×10⁶ cells/mL are seeded into T75 flasks containing between 5-30 mL culture medium, preferably between 10-25 mL culture medium, and most preferably about 10 mL culture medium. The cells are cultured until confluent, the medium is changed and in one embodiment the ACCS is collected 1 day post-confluence. In another embodiment the medium is changed and ACCS is collected 2 days post-confluence. In another embodiment the medium is changed and ACCS is collected 4 days post-confluence. In another embodiment the medium is changed and ACCS is collected 5 days post-confluence. In a preferred embodiment the medium is changed and ACCS is collected 3 days post-confluence. In another preferred embodiment the medium is changed and ACCS is collected 3, 4, 5, 6 or more days post-confluence. Skilled artisans will recognize that other embodiments for collecting ACCS from AMP cell cultures, such as using other tissue culture vessels, including but not limited to cell factories, flasks, hollow fibers, or suspension culture apparatus, or collecting ACCS from sub-confluent and/or actively proliferating cultures, are also contemplated by the methods of the invention. It is also contemplated by the instant invention that the ACCS be cryopreserved following collection. It is also contemplated by the invention that ACCS be lyophilized following collection. It is also contemplated by the invention that ACCS be formulated for sustained-release following collection. Skilled artisans are familiar with cryopreservation lyophilization, and sustained-release formulation methodologies.

The ACCS of the invention is characterized by assaying for physiologically relevant cytokines secreted in the physiologically relevant range of ˜5-16 ng/mL for VEGF, ˜3.5-4.5 ng/mL for Angiogenin, ˜100-165 pg/mL for PDGF, ˜2.5-2.7 ng/mL for TGF132, ˜0.68 μg mL for TIMP-1 and ˜1.04 μg/mL for TIMP-2.

It is also contemplated by the invention that ACCS, including pooled ACCS, be concentrated prior to use. The appropriate level of concentration required will be dependent upon the intended use and therefore will need to be empirically determined.

### Generation of PCS

A non-cellular derived form of CFC termed Physiologic Cytokine Solution (PCS) is generated by combining physiological levels of VEGF, Angiogenin, PDGF, TGFβ2, TIMP-1 and TIMP-2, in a carrier. The physiological levels for these cytokines are the same as those found in ACCS. Suitable carriers include normal saline, PBS, lactated Ringer's solution, cell culture medium, etc. Such compositions are suitable for cryopreservation, lyophilization, sustained-release formulation, and the like.

It is contemplated that PCS may be produced such that it contains more concentrated levels of the factors than those found in CFC, including ACCS, and that it may be subsequently diluted with appropriate diluent prior to use. Appropriate diluents include, without limitation, normal saline, PBS, lactated Ringer's solution, cell culture media, conditioned cell culture media, water, and the like. Such dilutions may be 1:2, 1:3, 1:4, 1:5, 1:10, 1:100, etc. The appropriate concentrations and dilutions required will be dependent upon the intended use and therefore will need to be empirically determined.

The compositions of the invention can be prepared in a variety of ways depending on the intended use of the compositions. For example, a composition useful in practicing the invention may be a liquid comprising an agent of the invention, i.e. CFC, including ACCS, or PCS, in solution, in suspension, or both (solution/suspension). The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form of an ointment, salve, cream, or the like.

An aqueous suspension or solution/suspension useful for practicing the methods of the invention may contain one or more polymers as suspending agents. Useful polymers include water-soluble polymers such as cellulosic polymers and water-insoluble polymers such as cross-linked carboxyl-containing polymers. An aqueous suspension or solution/suspension of the present invention is preferably viscous or muco-adhesive, or even more preferably, both viscous and muco-adhesive.

Sustained-Release Compositions

The CFS compositions, including but not limited to ACCS, pooled ACCS and PCS, may be formulated as sustained-release CFS compositions (referred to herein as “SR-CFS”). Skilled artisans are familiar with methodologies to create sustained-release compositions of therapeutic agents, including protein-based therapeutic agents such as ACCS, pooled ACCS or PCS. Details on creating SR-CFS compositions can be found in U.S. patent application Ser. No. 12/228,043, filed Aug. 8, 2008, the contents of which are incorporated herein by reference.

Aerosol Compositions

The methods for creating aerosol compositions is well known to skilled artisans. Specifics can be found in “Drug Delivery to the Lung” By Hans Bisgaard, Christopher O'Callaghan, Gerald C. Smaldone, published by Informa Health Care, 2001, and elsewhere in the scientific literature. Such methods are useful in creating aerosol compositions of ECS cells, including AMP cells, ACCS, PCS, etc.

Pharmaceutical Compositions of CFS Compositions

The present invention provides pharmaceutical compositions of CFS compositions and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the composition is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin, and still others are familiar to skilled artisans. Details on creating pharmaceutical compositions of CFS compositions can be found in U.S. patent application Ser. No. 12/228,043, filed Aug. 8, 2008, the contents of which are incorporated herein by reference.

Treatment Kits Comprising CFS Compositions

The invention also provides for an article of manufacture comprising packaging material and a pharmaceutical composition of the invention contained within the packaging material, wherein the pharmaceutical composition comprises CFS compositions. The packaging material comprises a label or package insert which indicates that the CFS compositions contained therein can be used for therapeutic applications such as, for example, healing inhalation injuries. Details on creating such treatment kits can be found in U.S. patent application Ser. No. 12/228,043, filed Aug. 8, 2008, the contents of which are incorporated herein by reference.

Formulation, Dosage and Administration of CFS Compositions

Compositions comprising ECS cells, including AMP cells, and CFS compositions, alone or in combination with each other and/or other agents, may be administered to a subject to provide various cellular or tissue functions, for example, to accelerate inhalation injury healing. As used herein “subject” may mean either a human or non-human animal.

Such compositions may be formulated in any conventional manner using one or more physiologically acceptable carriers optionally comprising excipients and auxiliaries. Proper formulation is dependent upon the route of administration chosen. The compositions may be packaged with written instructions for their therapeutic use. The compositions may also be administered to the recipient in one or more physiologically acceptable carriers. Carriers for may include but are not limited to solutions of normal saline, phosphate buffered saline (PBS), lactated Ringer's solution containing a mixture of salts in physiologic concentrations, or cell culture medium.

In addition, one of skill in the art may readily determine the appropriate dose of the CFS compositions for a particular purpose. A preferred dose is in the range of about 0.1-to-1000 micrograms per square centimeter of applied area. Other preferred dose ranges are 1.0-to-50.0 micrograms/applied area. In a particularly preferred embodiment, it has been found that relatively small amounts of the CFS compositions are therapeutically useful. One of skill in the art will also recognize that the number of doses to be administered needs also to be empirically determined based on, for example, severity and type of inhalation injury being treated. For example, in a preferred embodiment, one dose is sufficient to have a therapeutic effect (i.e. accelerate inhalation injury healing). Other preferred embodiments contemplate, 2, 3, 4, or more doses for therapeutic effect.

The skilled artisan will recognize that a preferred dose is one which produces a therapeutic effect (a therapeutically effective amount) such as accelerating wound healing, in a patient in need thereof. Of course, proper doses of the CFS compositions will require empirical determination at time of use based on several variables including but not limited to the severity and type of injury, disorder or condition being treated; patient age, weight, sex, health; other medications and treatments being administered to the patient; and the like. One of skill in the art will also recognize that number of doses (dosing regimen) to be administered needs also to be empirically determined based on, for example, severity and type of injury, disorder or condition being treated. In addition, one of skill in the art recognizes that the frequency of dosing needs to be empirically determined based on severity and type of injury, disorder or condition being treated. In certain embodiments, one dose is administered every day for a given number of days (i.e. once a day for 7 days, etc.). In other embodiments, multiple doses may be administered in one day (every 4 hours, etc.). Multiple doses per day for multiple days are also contemplated by the invention.

In further embodiments of the present invention, at least one additional agent may be combined with the CFS compositions. Such agents may act synergistically with the CFS compositions of the invention to enhance the therapeutic effect. Such agents include but are not limited to growth factors, cytokines, chemokines, antibodies, inhibitors, antibiotics, immunosuppressive agents, steroids, anti-fungals, anti-virals or other cell types (i.e. stem cells or stem-like cells, for example AMP cells). Inactive agents include carriers, diluents, stabilizers, gelling agents, delivery vehicles, ECMs (natural and synthetic), scaffolds, and the like. When the CFS compositions are administered conjointly with other pharmaceutically active agents, even less of the CFS compositions may be needed to be therapeutically effective.

The timing of administration of CFS compositions will depend upon the type and severity of the inhalation injury being treated. In one embodiment, the CFS compositions are administered as soon as possible after the injury. In another embodiment, CFS compositions are administered more than one time following injury.

CFS compositions may also be inserted into a delivery device, e.g., a nebulizer or atomizer or vaporizer, in different forms. For example, the CFS compositions can be part of a solution contained in such a delivery device. As used herein, the term “solution” includes a pharmaceutically acceptable carrier or diluent. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and may optionally be preserved against the contaminating action of microorganisms such as bacteria and fungi through the use of, for example, parabens, chlorobutanol, phenol, ascorbic acid, thimerosal, and the like. Solutions of the invention can be prepared by incorporating the CFS compositions in a pharmaceutically acceptable carrier or diluent and, as required, other ingredients enumerated above.

Exemplary Therapeutic Uses of CFS Compositions

Inhalation injury healing—The CFS compositions of the present invention are effective in accelerating healing of inhalation injuries caused by heat inhalation and/or damage from the inhalation of systemic toxins and/or damage from inhalation of irritant gases and/or damage from smoke inhalation. The instant invention is based upon the discovery that ECS cells, including AMP cells, and/or CFS compositions can accelerate the wound healing process for all wound types, particularly when administered topically, i.e. to the surface of the wound site. Using ECS cells, including AMP cells, and CFS compositions, all wound types undergo healing more rapidly than similar wounds left to heal naturally or which are treated with currently available methods. A “therapeutically effective amount” of a therapeutic agent within the meaning of the present invention will be determined by a patient's attending physician or veterinarian. Such amounts are readily ascertained by one of ordinary skill in the art and will enable accelerated healing when administered in accordance with the present invention.

### EXAMPLES

The following examples are put forth so as to provide those of ordinary skill in the art with a complete disclosure and description of how to make and use the compositions and methods of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees centigrade, and pressure is at or near atmospheric.

### Example 1

### Preparation of AMP Cell Compositions

Amnion epithelial cells were dissociated from starting amniotic membrane using the dissociation agents PXXIII. The average weight range of an amnion was 18-27 g. The number of cells recovered per g of amnion was about 10-15×10⁶ for dissociation with PXXIII.

Method of obtaining selected AMP cells—Amnion epithelial cells were plated immediately upon isolation from the amnion. After ˜2 days in culture non-adherent cells were removed and the adherent cells were kept. This attachment to a plastic tissue culture vessel is the selection method used to obtain the desired population of AMP cells. Adherent and non-adherent AMP cells appear to have a similar cell surface marker expression profile but the adherent cells have greater viability and are the desired population of cells. Adherent AMP cells were cultured in basal medium supplemented with human serum albumin until they reached ˜120,000-150,000 cells/cm². At this point, the cultures were confluent. Suitable cell cultures will reach this number of cells between ˜5-14 days. Attaining this criterion is an indicator of the proliferative potential of the AMP cells and cells that do not achieve this criterion are not selected for further analysis and use. Once the AMP cells reached ˜120,000-150,000 cells/cm², they were collected and cryopreserved. This collection time point is called p0.

### Example 2

### Generation of ACCS

The AMP cells of the invention can be used to generate ACCS, including pooled ACCS. The AMP cells were isolated as described above and ˜1×10⁶ cells/mL were seeded into T75 flasks containing ˜10 mL culture medium as described above. The cells were cultured until confluent, the medium was changed and ACCS was collected 3 days post-confluence. Optionally, the ACCS is collected again after 3 days, and optionally again after 3 days. Skilled artisans will recognize that other embodiments for collecting ACCS from confluent cultures, such as using other tissue culture vessels, including but not limited to cell factories, flasks, hollow fibers, or suspension culture apparatus, etc. are also contemplated by the methods of the invention (see Detailed Description above). It is also contemplated by the instant invention that the ACCS be cryopreserved, lyophilized, irradiated or formulated for sustained-release following collection. It is also contemplated that ACCS be collected at different time points (see Detailed Description for details).

### Example 3

### Generation of Pooled ACCS

ACCS was obtained essentially as described above. In certain embodiments, ACCS was collected multiple times from an AMP culture derived from one placenta and these multiple ACCS collections were pooled together. Such pools are referred to as “SP pools” (more than one ACCS collection/one placenta). In another embodiment, AMP cultures were derived from several placentas, i.e. from 5 or 10 placentas. The AMP cells from each placenta were cultured and one ACCS collection from each culture was collected and then they were all pooled. These pools are termed “MP1 pools” (one ACCS collection/placenta, multiple placentas). In yet another embodiment, AMP cell cultures were derived from several placentas, i.e. from 5 or 10 placentas. The AMP cells from each placenta were cultured and more than one ACCS collection was performed from each AMP cell culture and then pooled. These pools are termed “MP2 pools” (more than one ACCS collection/placenta, multiple placentas).

### Example 4

### Production of PCS

The following PCS compositions are produced:

Composition A: VEGF and TIMP-1; Composition B: VEGF, Angiogenin and TIMP-1; Composition C: VEGF, Angiogenin, PDGF-BB and TIMP-1; Composition D: VEGF, Angiogenin, PDGF-BB, TGFβ2 and TIMP-1; Composition E: VEGF and TIMP-2; Composition F: VEGF, Angiogenin and TIMP-2; Composition G: VEGF, Angiogenin, PDGF-BB and TIMP-2; Composition H: VEGF, Angiogenin, PDGF-BB, TGFβ2 and TIMP-2; Composition I: VEGF, TIMP-1 and TIMP-2; Composition J: VEGF, Angiogenin, TIMP-1 and TIMP-2; Composition K: VEGF, Angiogenin, PDGF-BB, TIMP-1 and TIMP-2; Composition L: VEGF, Angiogenin, PDGF-BB, TGFβ2, TIMP-1 and TIMP-2; Composition M: Angiogenin and TIMP-1; Composition N: Angiogenin, PDGF-BB and TIMP-1; Composition O: Angiogenin, PDGF-BB, TGFβ2 and TIMP-1; Composition P: Angiogenin and TIMP-2; Composition Q: Angiogenin, PDGF-BB and TIMP-2; Composition R: Angiogenin, PDGF-BB, TGFβ2 and TIMP-2; Composition S: Angiogenin, PDGF-BB, TGFβ2, TIMP-1 and TIMP-2; Composition T: PDGF-BB and TIMP-1; Composition U: PDGF-BB, TGFβ2 and TIMP-1; Composition V: PDGF-BB and TIMP-2; Composition W: PDGF-BB, TGFβ2 and TIMP-2; Composition X: PDGF-BB, TIMP-1 and TIMP-2; Composition Y: PDGF-BB, TGFβ2, TIMP-1 and TIMP-2.

VEGF, Angiogenin, PDGF-BB, TGFβ2, TIMP-1 and TIMP-2 are added at the following physiologic levels: ˜5-16 ng/mL for VEGF, ˜3.5-4.5 ng/mL for Angiogenin, ˜100-165 pg/mL for PDGF, ˜2.5-2.7 ng/mL for TGFβ2, ˜0.68 μg mL for TIMP-1 and ˜1.04 μg/mL for TIMP-2.

VEGF may be obtained from Invitrogen, catalog #PHG0144, PHG0145, PHG0146, PHG0141 or PHG0143; Angiogenin may be obtained from R&D Systems, catalog #265-AN-050 or 265-AN-250; PDGF-BB may be obtained from Invitrogen, catalog #PHG0044, #PHG0045, #PHG0046, #PHG0041, #PHG0043; TGFβ2 may be obtained from Invitrogen, catalog #PHG9114; TIMP-1 may be obtained from R&D Systems, catalog #970-TM-010; and TIMP-2 may be obtained from R&D Systems, catalog #971-TM-010.

VEGF, Angiogenin, PDGF-BB, TGFβ2, TIMP-1 and TIMP-2 are added to a carrier such as normal saline, PBS, lactated Ringer's solution, cell culture media, or other suitable aqueous solutions known to skilled artisans.

### Example 5

### Effects of ACCS in an Animal Model of Chronic Wound Healing

An art-accepted animal model for chronic granulating wound was used to study the effects of ACCS on chronic wound healing (Hayward P G, Robson M C: Animal models of wound contraction. In Barbul A, et al: Clinical and Experimental Approaches to Dermal and Epidermal Repair: Normal and Chronic Wounds. John Wiley & Sons, New York, 1990.).

Results: ACCS was effective in not allowing proliferation of tissue bacterial bioburden. ACCS allowed accelerated healing of the granulating wound significantly faster than the non-treated infected control groups.

### Example 6

### Use of ECS Cells, Including Amp Cells, and CFS Compositions, Including ACCS, in an Animal Model of Smoke Inhalation-induced Injury

An art-accepted animal model for combined burn and smoke inhalation-induced injury (Mizutani, A., et al., J Appl Physiol 105:675-684, 2008, incorporated herein by reference) is used to study the effects of the ECS cells, including AMP cells and CFS compositions including ACCS and PCS, on the healing of inhalation injury.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification

What is claimed is:

1. A method for treating inhalation injuries in a subject in need thereof comprising administering to the subject an effective amount of a composition selected from the group consisting of extraembryonic cytokine-secreting (ECS) cells, extraembryonic cell-derived cellular cytokine solution, physiologic cytokine solution (PCS), or a combination thereof.

2. The method of claim 1 wherein the inhalation injury is a thermal burn.

3. The thermal burn of claim 2 which is caused by heat or steam.

4. The inhalation injury of claim 1 which is caused by an irritant gas.

5. The irritant gas of claim 4 which is selected from the group consisting of sulfur dioxide, nitrogen dioxide, ammonia and chlorine.

6. The inhalation injury of claim 1 which is smoke inhalation.

7. The extraembryonic cell-derived cellular cytokine solution of claim 1 which is Amnion-derived Cellular Cytokine Solution (ACCS).

8. The composition of claim 7 wherein the ACCS comprises physiologic concentrations of VEGF, TGFβ2, Angiogenin, PDGF, TIMP-1 and TIMP-2.

9. The PCS of claim 1 comprising a therapeutic component consisting essentially of physiologic concentrations of VEGF, TGFβ2, Angiogenin, PDGF, TIMP-1 and TIMP-2, and a carrier, wherein the carrier is normal saline, PBS, lactated Ringer's solution or cell culture medium.

10. The method of claim 1 wherein the ECS cells are Amnion-derived Multipotent Progenitor (AMP) cells.

11. The compositions of claim 1 which are aerosolized.

12. The compositions of claim 11 which are administered by nebulizer, vaporizer, or atomizer.
